(19) United States
(12) Patent Application Publication
(10) Pub. No.: US 2013/0253283 A1
(43) Pub. Date: Sep. 26, 2013

(54) System and Method for Monitoring Central Nervous System Development

(71) Applicants: Bryan Hudson, Indianapolis, IN (US); Stephanie Peabody-Hudson, Indianapolis, IN (US)

(72) Inventors: Bryan Hudson, Indianapolis, IN (US); Stephanie Peabody-Hudson, Indianapolis, IN (US)

(21) Appl. No.: 13/428,331

(22) Filed: Mar. 23, 2012

(51) Int. Cl. A61B 5/00

(52) U.S. Cl. 600/300

(57) ABSTRACT

A system and method for monitoring treatment of central nervous system disorders of a patient. Initially, a plurality of patient symptoms are selected from a list of predetermined symptoms which correspond to NINDS common data elements. The symptoms are then measured in standardized units, and changes in the symptoms are recorded. The recorded changes are then standardized and an indication of the standardized changes is displayed. The above steps are and repeated until the monitoring, which may span the patient's lifetime, is concluded.

### BACKGROUND

In 2006, the Global Burden of Disease (GBD) study, a collaborative project of the World Health Organization (WHO), the World Bank and the Harvard School of Public Health, drew the attention of the international health community to the burden of neurological disorders and many other chronic conditions. The GBD report confirmed that neurological conditions are highly prevalent worldwide. The Global Burden of Disease report also drew the attention of the international health community to the fact that the burden of neurological disorders has been seriously underestimated by traditional epidemiological methods that took into account only mortality, but not disability rates.

The GBD study showed that over the years the global health impact of neurological disorders had been grossly underestimated. This report specifically showed that while the neurological disorders are responsible for about one percent of deaths, they account for almost 11 percent, and rising, of disease burden the world over. The GBD report has demonstrated that magnitude and burden of neurological conditions are huge and that they are priority health problems globally. The extension of life expectancy and the aging of the general populations in both developed and developing countries are likely to increase the prevalence of many chronic and progressive physical and mental conditions including neurological conditions.

With awareness of the massive burden associated with neurological conditions came the recognition that neurological services and resources were disproportionately scarce, especially in low income and developing countries. Furthermore, a large body of evidence shows that policy-makers and healthcare providers may be unprepared to cope with the predicted rise in the prevalence of neurological and other chronic conditions and the disability resulting from the extension of life expectancy and aging of populations globally.

In response to these findings, WHO and the World Federation of Neurology (WFN) recently collaborated in an international Survey of Country Resources for Neurological Disorders involving 109 countries and covering over 90% of the world's population. The survey collected information from experts on several aspects of the provision of neurological care around the world, ranging from frequency of neurological conditions to the availability of neurological services across countries and settings.

The findings show that resources are clearly inadequate for patients with neurological conditions in most parts of the world; they highlight inequalities in the access to neurological care across different populations, especially in those living in low-income countries and in the developing regions of the world.

### SOLUTION TO THE PROBLEM

The present system provides lifetime support, promoting CNS (central nervous system) wellness and prevention, and providing monitoring of CNS development in the pediatric population, as well as the aging CNS in the geriatric population. Monitoring programs in the present system also act as early classification/screening tools. Beyond the monitoring and screening of CNS development and aging, the present system provides for remote intervention/treatment of such CNS conditions as neurodevelopmental, acquired, neuromuscular, neurodegenerative, and headache. These common CNS occurrences represent a substantial component of the global burden of neurological conditions.

The system is designed to capture the National Institute of Neurological Disorders and Stroke (NINDS) Common Data Elements (CDE), necessary for multiple research purposes including CNS and pharma discovery. Through its data aggregation resources the present system is also designed to collect and disseminate training and evidence based resources, for utilization in CME (continuing medical education) as well as patient and care-giver training environments.

For caregivers, CNS treatment and monitoring may relieve stress and reduce the time spent on transporting family members back and forth from multiple clinicians offices and emergency rooms, reducing barriers to care and excessive consumption of care. In addition, it allows caregivers to be recognized as an integral part of the treatment team and process.

In an exemplary embodiment, the present system functions via cloud computing. Providers can pay as they go, and, therefore, operating costs can be better managed over time. A cloud computing architecture enables providers to link disparate systems from different organizations and scale up the program as it grows. This ensures an always-on capability that is crucial for health-related applications. The present system is designed to provide services to all CNS patients, including neurodevelopmental and neuropsychiatric, etc., services across the patients' lifespan.

Very specific data is collected related to treatment (CDE's as well as primary outcome data). Once patient is discharged, the present system allows for ongoing connection. The system represents a lifespan model of health care service delivery that is intended to promote CNS wellness and prevention, promote early identification of CNS concerns in the primary care setting, provide chronic CNS disease management, collect real-time medical and health care data to promote CNS innovations and discoveries related to diagnosis, treatment, and prevention.

The present system supports secure exchange and sharing of patient health data across multiple health information exchange systems. In an exemplary embodiment, the system comprises an interactive, cloud-based portfolio of integrated health applications that allow for the collection and incorporation of patient data/records from multiple sources into a standard, user-friendly viewing and communication tool. A HIPPA secure, clinical care management system provides real-time access and connection for clinicians, the network of health care providers, family caregivers, and the patient to information about the patient's diagnosis, treatment, treatment outcomes, and education relevant to the patient's condition.

The present system and method transforms the delivery of CNS prevention, wellness, clinical treatment, monitoring, diagnostics and care by maximizing the use of technology to help connect all members of a person's healthcare delivery system across his/her lifespan. The system also improves access to medical and educational resources, training and services, while reducing costs and ultimately improving patient outcomes and quality of life.

### BRIEF DESCRIPTION OF THE DRAWINGS

FIG. 1 is a system diagram showing exemplary computer hardware and connectivity thereof in one embodiment of the present system;

FIG. 2A is a flowchart showing an exemplary set of steps performed in execution of the present method;

FIG. 2B is a diagram of an exemplary developmental profile;

FIG. 2C is a continuation of FIG. 2A;

FIG. 3A is a flowchart showing an exemplary set of steps performed in accordance with the master treatment template;

FIG. 3B is a diagram of one exemplary trend analysis graph;

FIG. 4 is a diagram of an exemplary master treatment graph; and

FIG. 5 is a flowchart showing an exemplary set of steps performed in accordance with the master diagnostic template.

### DETAILED DESCRIPTION

### Overview

The present method and system comprises a computer-implemented system for monitoring central nervous system (CNS) development, and treating CNS-related issues over a patient's lifetime. The present method and system functions to provide for the chronic, acute, post acute and rehabilitation aging populations by collecting health data, evaluating the data, and aggregating the data. Longitudinal, real-time data collection and analysis for outcome measurement, research and discovery is also performed by the system.

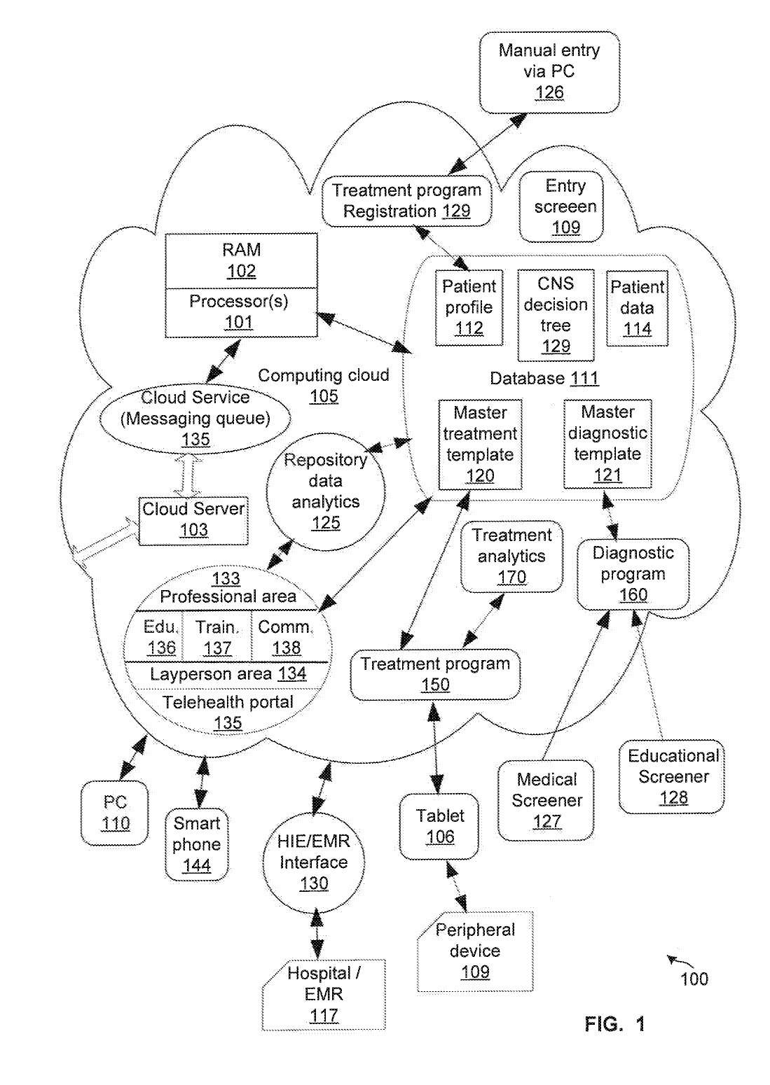

FIG. 1 is a system diagram showing exemplary computing and data storage devices and the connectivity thereof in one embodiment of the present system 100. As shown in FIG. 1, in an exemplary embodiment, system 100 is Internet cloud-based, using computing cloud 105 as a communication and storage medium. Cloud computing typically involves multiple cloud components communicating with each other, and with external devices, over a loose coupling mechanism. In an exemplary embodiment of the present system, this coupling mechanism comprises a messaging queue 135 coupled to a cloud server 103.

System 100 includes one or more processors 101 with associated RAM (and/or other types of) memory 102, and a database 111 in which is stored data and applications including a plurality of patient profiles 112, patient data 114 for each patient being treated through the present system, a plurality of different types of decision trees 129 (only one is shown in FIG. 1), master treatment template 120, and master diagnostic template 121.

Processor 101 and/or other cloud-connected processors (not shown) execute(s) the various computer applications/programs shown in FIG. 1 inside of computing cloud 105. Cloud 105 provides access to database 111 for these applications including treatment program registration 129, repository data analytics 125, treatment program 150, diagnostic program 160, and treatment analytics 170. These applications are described in detail below.

A variety of peripheral devices and applications may be connected to the cloud-based resources in system 100, including a PC (personal computer or equivalent device) 110, a smart phone 144, a tablet 106, medical screener application 127, and educational screener application 128. HIE/EMR (Health information exchange/Electronic Medical Records) interface 130 provides system access to hospital and electronic medical records 117. Medical and educational screener applications 127 and 128 may use any suitable Internet-connected device (e.g., a PC) to communicate with database 111 and applications in cloud 105.

The present system provides an interactive prescription and medication management pathway, via HIE/EMR interface 130, between an ordering healthcare practitioner and providers of lab results, radiologic readings and images, and other diagnostic tests. The prescription and medication management pathway connects the medical practitioner and patient's pharmacy to swiftly order prescriptions and provide necessary documentation.

Telehealth portal 135 (which there may be more than one of) may be used by professionals (via ‘professional’ portal 133) and non-professionals (via ‘layperson portal 134) in areas including education, communication, and training. An education sub-portal 136 allows for the accumulation and dissemination of literature specific to a particular patient or patients with similar conditions. Professionals may use this data for purposes of further understanding the condition to enhance treatment. Laypersons might use this data for purposes of learning more about the patients conditions to enhance their interaction with and treatment of the patient. The primary differences between data located in the professional and layperson education areas is that the former will typically include literature from peer reviewed journals or from the physician or professional provider written for a medical professional, whereas the non-professional data includes literature written for a lay group/non-medical professional. Both areas allow for uploading data pertinent to the audience in question. Therefore, a medical provider might upload an easily readable summary of some journal article into the non-professional area. Likewise, a non-professional might upload a narrative about a patient based on their perspective.

Thus an open forum is provided where information that may be helpful to a patient can be made available, in language/vernacular applicable to the audience being addressed. The data accumulated for a particular patient may be placed in a general library for others to use, for example, a library cataloged by specific conditions can be generated from the data.

A training sub-portal 137 includes access to a store of certification courses and continuing education courses that are applicable to either a medical professional or non-medical professional. These courses and training are either public domain or licensed to the company for use for the purpose of offering continuing education or certification. The purpose of this portal is to provide additional functionality for the software as well as to provide some certification and education possibilities to providers who treat CNS disorders.

A communication sub-portal 138 provides non-private communication between the medical and non-medical providers. This portal operates in manner similar to that of social media such as Facebook®. Messages may be left for a designated individual or the treatment group as a whole and can be responded to by anyone on the treatment team reading the message. The purpose behind this format is to allow everything being discussed regarding the patient is open to all involved with the patient's care. Data in the communication portal may be destroyed upon patient discharge from the system.

Entry points for the Treatment Program

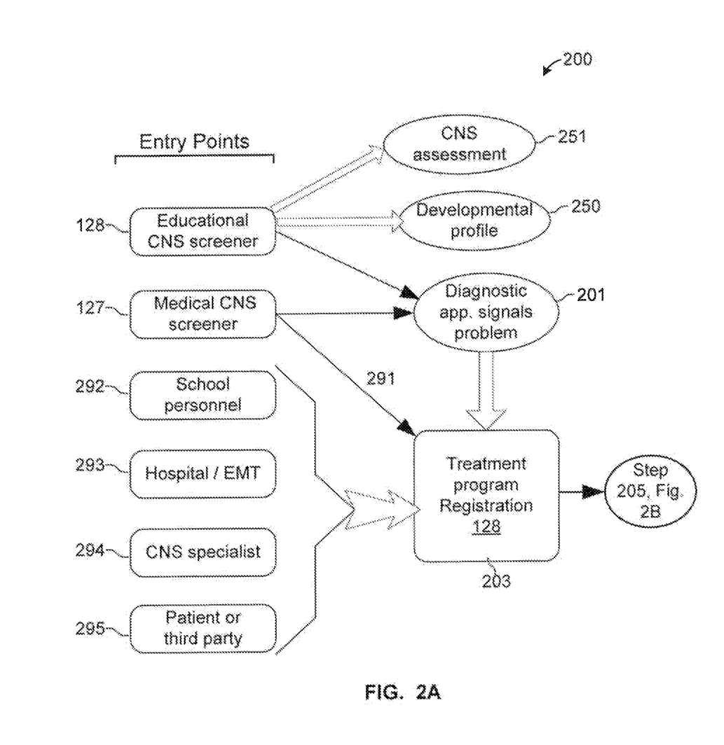

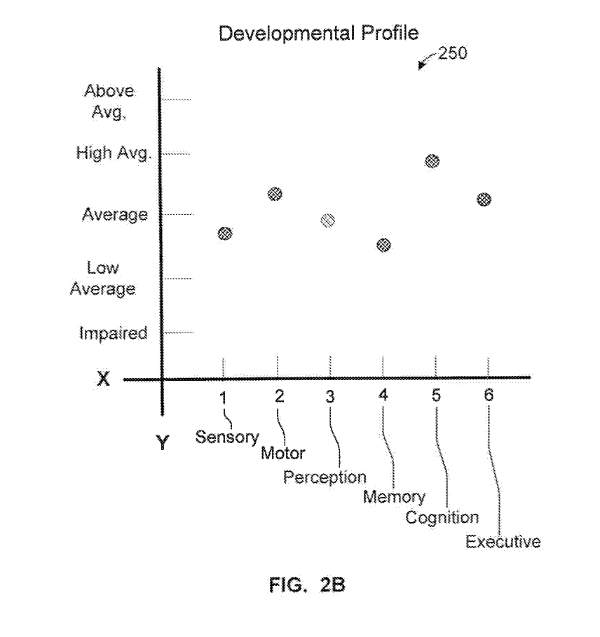

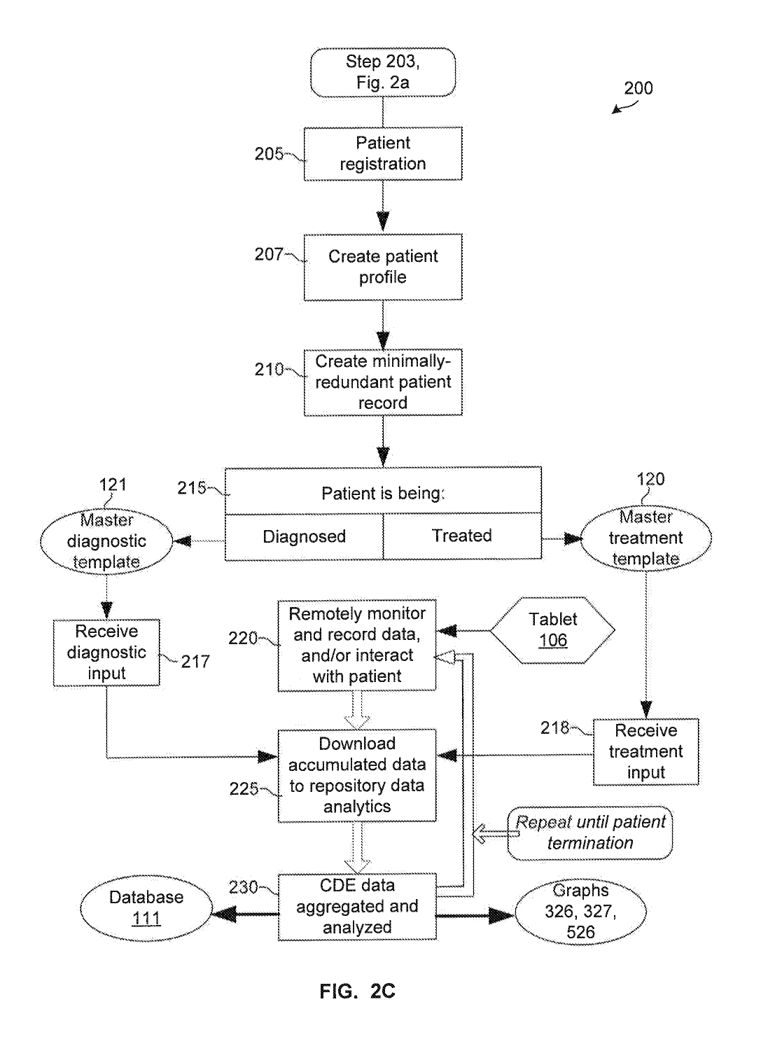

FIG. 2A is a flowchart showing an exemplary set of steps performed in initial execution of the present method. As shown in FIG. 2A, a teacher, special education coordinator, or other school personnel may request that the patient take an educational CNS diagnostic screener 128, which comprises a series of age-appropriate CNS questions designed to uncover the earliest sign of CNS disorders. Upon detecting a potential problem at step 201, the patient is entered in the present CNS treatment system (described in steps 201-230. FIGS. 2A and 2B). Using this option, the school personnel would be a primary provider, however at some point it is expected that the patient's actual primary care provider (PCP) and possibly other medical professionals would contribute to the treatment and/or monitoring of the patient's identified condition.

Educational CNS screener 128 also maintains a record of developmental functioning regarding the central nervous system. While most screeners focus on pathological factors (e.g., whether a person has some particular symptom or not), the present system focuses on developments that should be occurring at different age periods, and deviation from this normal pattern represents a ‘signal’ for potential problems. Moreover, in that it is normally initiated at the first, or close to the first, well-baby check-up, the present system provides a developmental CNS history of an individual over time in a standardized, age-appropriate format. Educational CNS screener 128 (FIG. 1) generates results including a snapshot developmental profile 250 of CNS functioning (starting preferably from birth, or whenever treatment is started, with snapshot data (data displayed for a specific point in time) continuing until death or other program termination. Screener 128 also generates a standardized, age-appropriate CNS assessment 251 for each episode in which screener 127 or 128 is administered.

FIG. 2B is a diagram of an exemplary developmental profile 250. In one embodiment, the developmental profile 250 is in the form of a graph indicating how the subject, for his or her age, compares to others the same age with respect to the measures used by the screener. In the example of FIG. 2B, a point (a cross-hatched circle) is displayed for each of six CNS-related functions including sensory (1), motor (2), perception (3), memory (4), cognition (5), and executive (6), indicating a range between low average and high average for the respective functions.

Educational CNS screener 128 (as well as medical screener 127 described below), is embedded in (or called from) the central CNS program 109 (FIG. 1), which is a cloud-based entry screen program that allows a user to initiate diagnosis of a CNS condition based on the results of the screener as well as initiate treatment for a suspected condition. This precautionary ability of the present system allows it to identify CNS conditions that are first witnessed in the classroom, so that formal diagnosis or treatment may begin immediately, as opposed to the traditional educational model which may take between three to six months (half of a school year) to identify and initiate treatment.

Central CNS program 109 links all of the applications in the present system together, and an entry screen program responds to requests from other applications by calling the appropriate application and passing requestor data. While any of these applications may be accessed directly through the entry screen program, tabs for each of the other programs are also available on each program. Therefore, if one entered the present CNS program through treatment program 150, the person then also has access to other applications/programs via tabs on a screen displayed by treatment program 150. These tabs lead the user back to the entry screen where the desired selection is made available. No matter where a user logs in from, the entry screen and a menu of services, accessible via the tabs, is displayed on the user's device.

Medical and educational screener programs 127/128, like diagnostic and treatment programs 160/170, are stand-alone software applications that are linked to the diagnostic and treatment programs. In the event that a screener program indicates a problem, the provider employing the screener can go to the treatment or diagnostic program by using tabs designated as “treatment” or “diagnostic” in the respective screener application 127 or 128. Alternatively, a patient might decide not to use either the treatment or diagnostic software and instead simply use the screener to find out what action is recommended. In either case the screener data is downloaded into repository data analytics 125 under that patient's unique identification code.

### Example Questions: In the Educational CNS Screening Process

1. Does the student daydream excessively (staring off into space)?

2. Has the student ever been non-responsive to his/her name or other verbal commands?

3. Has the student experienced unprovoked episodes of agitation or anger?

The above three questions, if answered in the affirmative, would signal the possibility of petite-mal seizures or seizures that are manifested in behaviors, as opposed to convulsions. In this situation, treatment program 150 may indicate, for example, that petite mal seizures would be in the differential of conditions that are causing these symptoms. Depending on how other screening questions are answered, there may be other differential diagnosis that need to be considered.

During a check-up at a PCP, the patient and PCP can enter the treatment program through participating in the medical CNS screener 127, which comprises a series of age-appropriate CNS questions similar to those in the educational CNS screener 128. In an alternative embodiment, simply completing the screener will register the patient in the CNS system, as indicated by arrow 291

A teacher, nurse or other school personnel 292 can enter a student into the CNS system if the student is found to have a CNS system that affects his or her functioning in the school setting or if the patient returns to school after being diagnosed with a CNS condition that will affect his functioning in school.

A patient may be entered into the CNS treatment program at the point of injury (by a first responder) 293 by accessing the program through their receiving hospital's Electronic Medical Records and entering data about the patient. The patient may, alternatively, be entered into the system upon entering the emergency room or after being admitted to the hospital secondary to a CNS condition.

The patient may be entered into the CNS treatment program after a visit with a CNS specialist or any specialist 294 that determines that the patient has a CNS disorder that requires ongoing treatment or monitoring. The parent, spouse, or other non-medical-professional third party, or the patient himself 295, may also initiate entry into the present system.

At this point (step 203), treatment program registration is effected. Entry via points 292-295 is made via a PC or other computing device 126 connected to computing cloud 105. Note that with the exception of entry point 293, all of the above-described entry points are novel, as currently e-treatment platforms are normally entered only in the hospital setting.

### Treatment Program

FIG. 2C is a continuation of FIG. 2A, showing an exemplary set of steps performed in execution of the present method. As shown in FIG. 2C, at step 205, once the present program has been initiated, a provider ID and user login password are provided as well as permissions for use of various aspects of the program. For example, a teacher or parent entering the program would not have access to the prescription capabilities of the program and while they could request a referral to a medical specialist, the referral would have to be made by a licensed medical provider. System clearance is performed via interaction with the CNS platform with the electronic records of the state licensing boards and hospital records pertaining to providers.

At step 207, the patient's profile 112 is then created or validated by the system. This includes registration of the patient, and while the initial data might have already been entered via one of the aforementioned entry points, a certified record is not made until this point.

Most patients will have a medical record at the hospital wherein the system is activated, or at another hospital. At step 210, HIE/EMR (Health information exchange/Electronic Medical Records) interface 130 communicates with an existing EMR system 117 in a reciprocal or one-way fashion, depending on the EMR system, to prevent redundant data entry. This step creates a patient record with minimal redundancy.

HIE/EMR interface 130 allows clinicians to access their hospitals' medical records and download them into database 111. This process diminishes the redundancy involved in the clinician having to enter the same data twice. In an exemplary embodiment, a clinician may enter a hospital's unique HIE number to provide access through their gateway and then receive the patient's current record including demographics, history, etc.

At step 215, data is entered into treatment program 150 regarding symptoms being treated or monitored and the process of data accumulation, aggregation, and standardization begins. This step represents the first use of the treatment aspects of the present program, which may take place at the school, home, hospital, practitioner's office, etc. At step 215, if a patient is being diagnosed, then control is passed to the master diagnostic template program 121, and diagnostic input is received from the patient at step 217. If the patient is being treated, then control is passed to the master treatment template program 120, and treatment input indicating the patient's symptoms being treated is received at step 218. System operation then continues at step 225, described below.

The present treatment program allows a clinician to select from a symptom list generated by the program or manually enter unique symptoms to be treated. These symptoms are then categorized into one of five categories—physical, cognitive, behavioral, social, and comorbid. This occurs before the master treatment template 120 (described below) is employed, as data from this step provides the categories and symptoms required by the master treatment template.

Regardless of where the present treatment program is initiated, ultimately, the patient will subsequently access the program from home. Once home, the patient and his or her caregivers may interact with the medical team remotely through computing cloud 105 and treatment program 150 either by use of their personal computer or with a tablet 106 or the like.

At step 220, the provider uses a peripheral device such as a ‘tablet’ 106 or the like to monitor and record certain biometrics and provide peripheral management. Tablet 106 may be any portable Internet-capable communication and display device such as an I-pad®, Android®, Vocare® pad, etc. Tablet 106 may, alternatively, be a personal computer (PC) or other suitable device. A tablet is specified because a tablet-type device is preferable when there is a need for input from biometric patient-monitoring peripherals.

In the present system, peripheral management comprises the use of one or more peripheral devices 109 that can measure biometric data, such as blood pressure, heart rate, blood sugar, etc. The present system allows for real time measurements and monitoring by a remote clinician via the transmission of this data via a tablet (or PC) 106 that can accept the data (e.g., a tablet or computer which is Bluetooth®-equipped or equipped with USB input channels). The transmitted data is placed in the master treatment template 120 in a special section labeled, e.g., “biometric input”. Under this section, all of the biometric data, if any, is recorded every time a patient inputs biometric data via the peripheral device. This peripheral management provides the capacity to record the trend of the changes in whatever is being measured (e.g., blood pressure over one week, one month, etc.), as described further below.

Once the patient has returned home, depending on their age, they may at some point go back to school or work. In either case the personnel directly responsible for the patient in these environments have the capability of joining the treatment team, if such is indicated and if they receive formal invitation from the medical provider or caregiver. If the teacher or work personnel are invited into the treatment team, like the parent or caregiver, they can provide input remotely.

At step 225, data accumulated from the program is systematically downloaded into the repository data analytics area 125 based on a set of defined parameters. For example, research data may consist of all Common Data Elements (CDEs) responded to by the medical team about the patient. Trend data related to treatment effects is also transferred to the repository data analytics area 125. Telehealth portal 135 uses data from repository data analytics storage 125 for various purposes.

At step 230, CDE data is aggregated and analyzed via the processes described below, including trend analysis and generation of master treatment graph 400, trend analysis graph 327, and master diagnostic graph 526. The aggregated data is stored in database 111. Steps 220 through 230 are then repeated until patient termination.

### Trend Analysis

Each symptom, clinician or other treatment provider, treatment outcome, and relevant CDE data is periodically aggregated, e.g., weekly, and sent to a patient data repository 114 in database 111. If a treatment is changed in type or quantity (decreased or increased) an automatic upload of the data designating the change will be performed by the system. The data once aggregated is subjected to trend analysis.

One important aspect of the present method for chronic disease management is in the ability to monitor disease trends in association with distinct correlated variables. The chronic disease management is part of the present treatment platform. The trending referred to is the result compiling data concerning a patient's treatment over time and then subjecting it to a formal trend analysis. An exemplary trending formula, embeded in most statistical programs, is in the form of:

(=Trend(A1;A2;A3:C2;C3;C4;C5;C6)

where A represents the symptoms of interest and C represents the different timeframes (week 1, 2, 3, etc.). The formula results in a line with a slope which is a measure of change (positive or negative) over time. New data points may be added to the trend line over time to generate a new slope or trend. Microsoft Excel®, for example, has a version of the above trending formula.

This trend analysis also provides a forecast of treatment results in the event that treatments and outcomes remain on the predicted slope, which allows for more refined decisions to be made about the continuation or changing of a treatment.

### Master Treatment Template

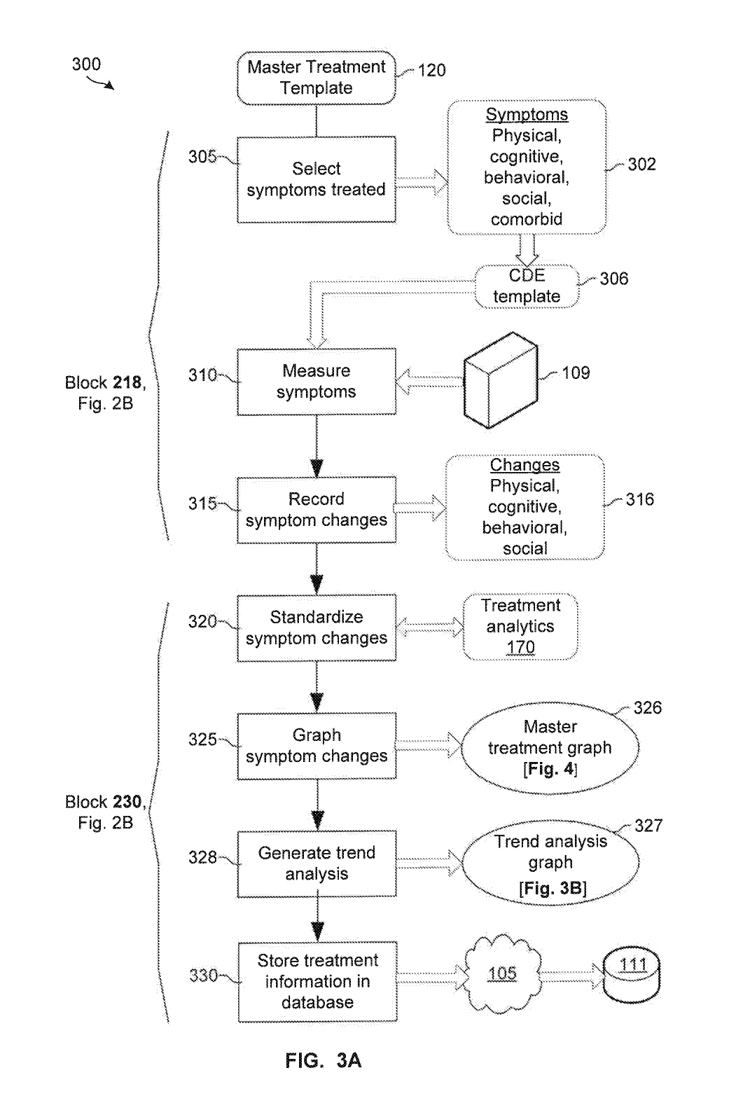

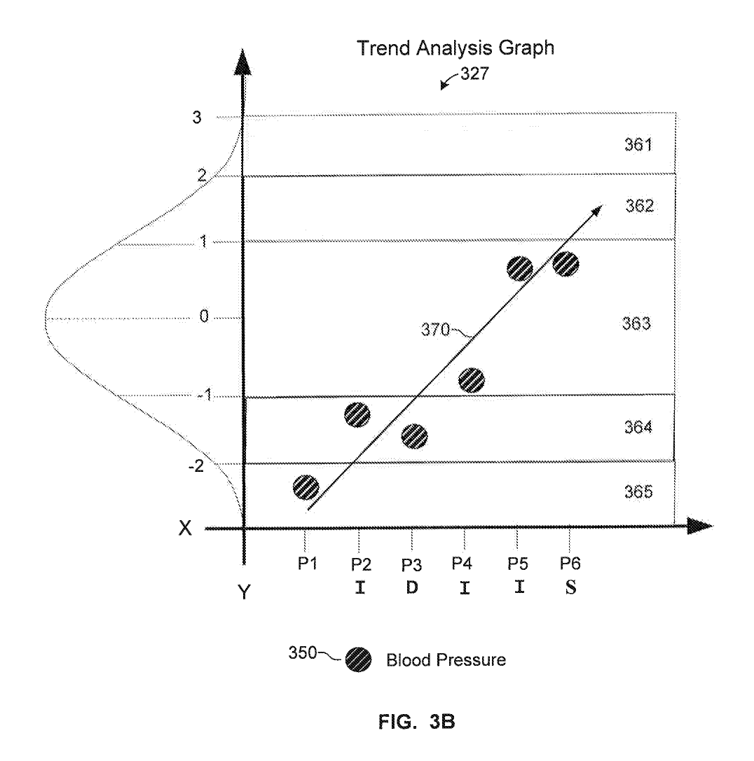

FIG. 3A is a flowchart 300 showing an exemplary set of steps performed in accordance with a master treatment template 120. Master treatment template 120 is a software application that can accommodate input from a large number of different clinicians and place that input into a single standardized graph, hereinafter termed a ‘master treatment graph’ 400 (described below).

As shown in FIG. 3A, at step 305, the symptoms to be treated are selected from a predetermined list of symptom categories 302, through use of a CDE template 306, which is a computer application that filters through only predetermined CDE-compliant types of data, which include physical, cognitive, behavioral, social, and comorbid symptoms. Next, the selected symptoms input from a peripheral device 109 and are measured, at step 310. At step 315, any changes in the symptoms in the areas of physical, cognitive, behavioral, social, and comorbidity are recorded after comparing the present measurement with the previously recorded measurement of the same symptom.

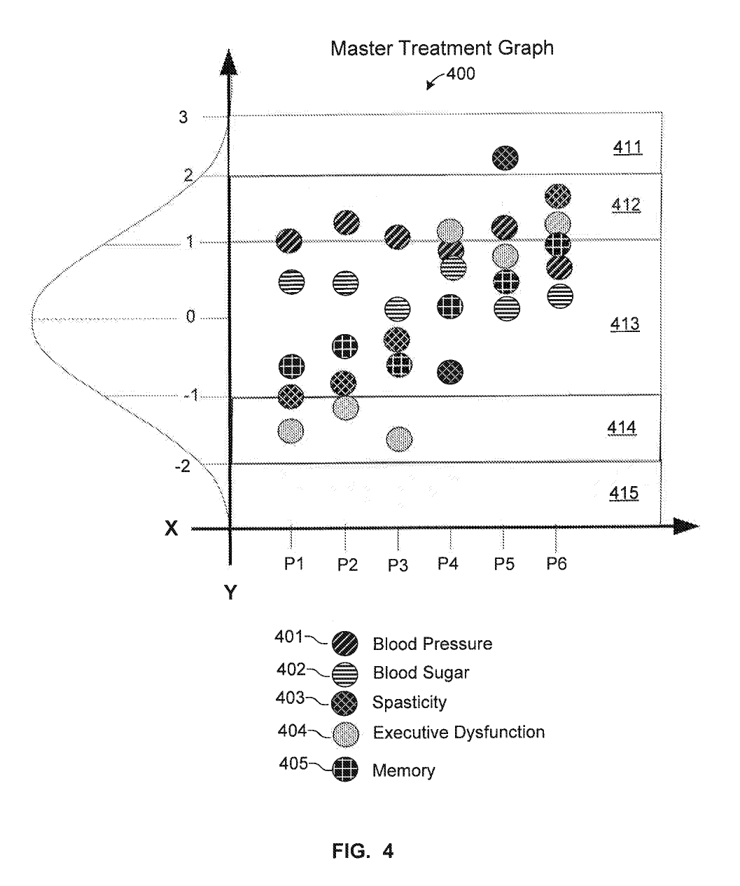

At step 320, any observed changes in any of the selected symptoms are standardized by treatment analytics application 170 which uses the input from various sources (providers) that report their findings in different ways. At step 325, the observed symptom changes (and any unchanged readings) are displayed on a master treatment graph 400 (described in detail in FIG. 4).

Master treatment template 120 not only standardizes data, it also generates, at step 328, a trend analysis graph 327 of the data over a period of time. At step 330, treatment information including currently measured symptoms, symptom changes, and other data generated for display on graphs 400 and 327, is stored in appropriate areas of database 111 via cloud 105.

FIG. 3B shows an exemplary trend analysis graph 327 of a patient's blood pressure over a period of time. Trend analysis graph 327 provides a view of the patient's symptoms over a selected period of time, showing how well the treatment is working in terms of, for example, the categories of “improved”, “stable”, or “declined”, as respectively indicated by one of the letters “I”, “S”. or “D”, below each time period (or time point) on the graph.

As shown in FIG. 3B, time points P1-P6 on the X axis respectively indicate the date at which patient data was entered. A trend analysis may performed on each symptom, over the time period of data entry (e.g., P1-P6) to determine if a particular symptom is trending toward improvement, decline, or stabilization. Each symptom may be subjected to this analysis depending on the provider's preference.

In the example of FIG. 3B, each hashed circle 350 in trend analysis graph 327 represents a patient's blood pressure reading at successive points in time, indicated by x-axis points P1-P6. As indicated in the FIG. 3B graph, the readings taken at points P2-P6 are indicated as being “I” (improved), “D” (declined), “I, “I”, and “S” (stable), respectively. Line 370 indicates the trend for this particular type of data. Trend analysis graph 327 is demarcated by symptom category so that it is layered, with each layer preferably color-coded (indicated by shading in FIG. 3B) to represent a different symptom category. The graphing shows whether the symptom's standardized score indicates above average (layer 361), high average (layer 362), average (layer 363), mild impairment (layer 364), or moderate to severe impairment (layer 365).

Master treatment template 120 lists all of a patient's providers on a drop-down window. Selecting the provider generates a result indicating the relationship between the provider (i.e., cardiologist, neurologist, parent, etc.) and the patient, what the provider is treating and how, and the results of the treatment (improved, stable, declined).

The master treatment template 120 also indicates all of the symptoms being treated and what treatment is being utilized. A drop-down window reveals the symptoms and once a symptom is selected, the person treating it and its reaction to treatment is demonstrated.

In addition, master treatment template 120 lists the treatment types via a drop-down window. When a treatment is selected, the symptom being treated and the effect of the treatment (Improved, stable, declined) is demonstrated, as well as which individuals are treating symptoms falling within this category. The master treatment template has a tab that includes all of the symptoms being measured by direct input (via a peripheral device 109).

All of the data collected about a symptom's improvement, stabilization, or decline is channeled into a treatment analytic program 170 which subjects each data point to a z-score transformation. Each symptom category will have zero or more symptoms transformed into “z-scores”. A z-score indicates how many standard deviations an observation or datum is above or below the mean for each data point, which represents the change in a particular symptom.

Treatment analytic program 170 initially transforms data entered by providers into a standardized language through the use of a ‘z-score’ transformation. This z-score transformation is used by the master treatment template 120. As described above, these z-scores are subjected to a trend analysis to determine how a particular treatment is performing over time. Therefore, the z-scores are used for ‘real time’ evaluation of a treatment. whereas the trend analysis allows for an overview of a treatment over a period of time.

As an example, consider a patient with a CNS condition who also has blood sugar problems which are being treated via medication. The symptom's changes (i.e., improvement or decline) are monitored daily, monthly, and weekly. The daily changes are transformed into z-scores which are then plotted as either improved-decline- or stable via color coded graph. These z-scores are then included with previous z-scores to determine the trend of treatment over time. Either the independent z-scores or trend analysis is available for view. If the treatment is not performing to expectation and is changed, this starts the process over, obtaining the first z-score for the forthcoming trend analysis.

### Master Treatment Template Features and Processes

Initially, a master treatment template 120 lists all of a patient's providers on a drop-down window. Selecting the provider shows the provider's relationship with the patient (i.e., cardiologist, neurologist, parent, etc.), what the provider is treating and how, and the results of the treatment (improved, stable, declined).

Template 120 also provides information concerning all of the patient's symptoms being treated and what treatment is being utilized. A drop-down window reveals the symptoms and once a symptom is selected, the person treating it, and the patient's reaction to treatment is displayed. Template 120 has a tab that displays all of the symptoms being measured by direct input via a peripheral device 109. This tab thus demonstrates the use of machine-entered data.

Master treatment template 120 also displays a list of treatment types via a drop-down window. When a particular treatment is selected, the symptom being treated and the effect of the treatment (Improved, stable, declined) is indicated, as well as which individual(s) are treating symptoms falling within this treatment category.

Data collected about each symptom's improvement, stabilization, or decline is channeled into the treatment analytics program 170 (step 320, FIG. 3) which subjects each data point to a z-score transformation, as explained below. Each symptom category then has zero or more symptoms transformed into z-scores.

FIG. 4 is an exemplary diagram showing master treatment graph 400. Master treatment graph 400 is generated by treatment analytic program 170 (step 325, FIG. 3). The graph 400 is demarcated by symptom category so that it is layered, with each layer preferably color-coded (indicated by shading in FIG. 4) to represent a different symptom category. In the example shown in FIG. 4, five symptoms—blood pressure (indicated by symbol 401), blood sugar (symbol 401), spasticity (symbol 403), executive dysfunction (symbol 404), and memory (symbol 405) are graphed over the period from time point P1 to time point P6. The graphing shows whether each symptom's standardized score indicates above average (layer 411), high average (layer 412), average (layer 413), mild impairment (layer 414), or moderate to severe impairment (layer 415).

Subsequent graphing reveals whether a symptom has improved or declined. The degree to which improvement, stabilization, or decline is demonstrated can be determined by clicking on the symptom in the graph which will show the last three measurements of the symptom in the original graphics (e.g., blood pressure in diastolic and systolic measurements as opposed to z-scores).

Master treatment graph 400 illustrates functions that have previously not been graphed in the same context (or on the same graph), because, for one reason, the graph includes input from sources including professionals, non-professionals, and non-medical professionals, for example, the report of treatment by a spouse, which may not appear on a typical medical record. The particular compilation of data in graph 400 is of obvious importance to the medical community.

When all of the parameters 401-405 are placed together on a graph 400 (or other chart), they represent a holistic picture of the patient's functioning that is not available in any previous medical record in any systematic fashion. The calculations necessary to put all the parameters in a standardized/simplified language represent a z-score transformation. In an exemplary embodiment, the standardized/simplified language uses terms such as “above average”, mild impairment”, and “moderate to severe impairment”, that are easy for all to understand while maintaining the quantitative validity of the original metrics, thus allowing for color-coding based on the status of the symptom. This manner of presentation provides immediate visual information about how the patient's CNS health in the environments that are significant (i.e., home, school, work) and not simply based on response to a particular medication or based on how a patient appears in an office visit.

Each symptom, treatment personnel, treatment outcome, and relevant CDE data is aggregated periodically, e.g., weekly, and sent to patient data repository 114. If a treatment is changed in type or amount (decreased or increased), an automatic upload of the data designating the change is performed by the system.

### Master Diagnostic Template

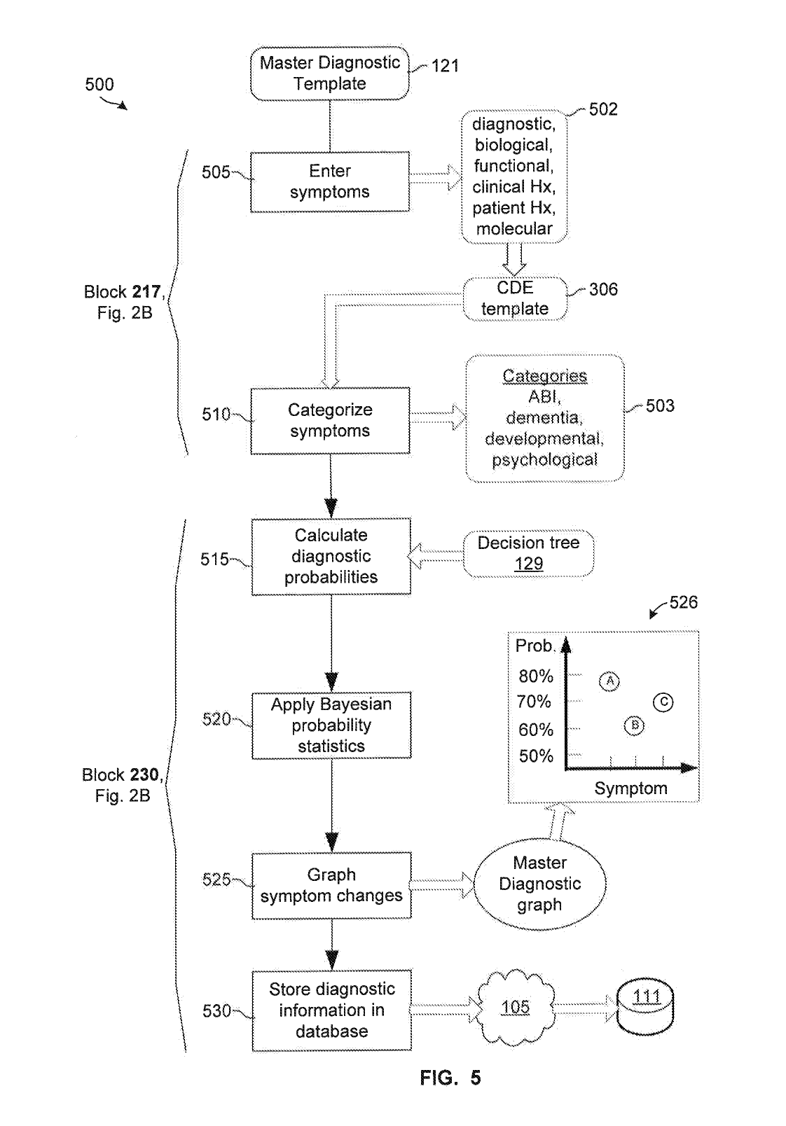

FIG. 5 is a flowchart showing an exemplary set of steps performed in accordance with a master diagnostic template 121. Master diagnostic template 121 is a software application that works in a fashion similar to that of the master treatment template 120, and provides a probability statistic via diagnostic probability graph 526 for each of the data points, related to some diagnostic possibility (e.g., patient history, family history, symptoms, lab results, etc.), that is entered. Master diagnostic template 121 standardizes the input into a common language and provides a quantitative analysis of these inputs in terms of a probable diagnosis.

As shown in FIG. 5, at step 505, symptoms from one of more categories 502 including some or all of diagnostic, biological, functional, clinical medical history, patient social history, and molecular, are entered into the master diagnostic template 121 via CDE template 306.

### Data Standardization

For each patient for which the present treatment program is initiated, CDE-compliant data is entered by a provider. One purpose of using CDE data is to set a standard and then determine how many people treating a particular condition are compliant with this standard. Thus, the data entered into the treatment programs is filtered through CDE template 306 only if it is one of the predetermined CDE-compliant types of data.

Some of this CDE data is demographic (e.g., age, race, etc.), and some of it is related to specific symptoms and treatments. Each CDE data template 306 is linked to the master treatment template symptom and treatment pull-down windows. Thus, if a CDE symptom is checked on the master treatment template, it will automatically be filled in, in the background, from the data entered by the provider.

For example, when a provider enters the age and race of the patient, since these are also CDE data, they will populate the CDE template 306 automatically. CDE template 306 accepts only data that has been designated on the master treatment template 120 as a CDE data point. The same is the case with symptoms and treatments. If, for example, inter-cranial pressure (ICP) is a symptom selected to be treated by the provider, this information will be accepted by the CDE template because ICP is a CDE. Moreover, the manner in which a symptom is treated will also be accepted by the template if the treatment selected is a CDE. If the treatment selected is not a CDE, it will not be entered into the system, and this part of the CDE for this patient will remain blank. While providers have previously been asked to collect these types of data, there has previously been no standard method of collection of this kind of data.

The present system address the above issue by essentially accepting only CDE-compliant data. This does not mean that a particular provider dealing with a patient must completely fill all of the CDE blanks on a CDE template. There will normally be some data that is not completed because the provider might choose to treat a symptom in a way that is not listed as a CDE. However, the present system compiles data on the frequency with which the standards suggested by the CDEs are actually followed, which is valuable data in its own right.

### Example of Data Standardization

The case scenario described above may be used as an example of how the present standardization process works. In the above scenario, each of the disturbances experienced by the patient (TBI, language, motor, and blood sugar problems) has a different metric used to report the extent of the problem. Language measures use a standard score based on a mean or average of 100+/−15 or 85-115. Blood sugar is measured in milligrams per deciliter with the average or acceptable range falling between 70 to 120 mg/dL, and motor disturbances are measured in terms of strength or tone, with the strength of a particular muscle (usually measured as peak isokinetic torque in Newton-meters) divided by body weight (usually measured in kilograms) and divided again by body height (usually measured in meters).

Ultimately, each specialty or clinician reports data in the customary or standard form of their discipline. However, even if a clinician attempts to report a qualitative summary of their findings (e.g., Poor, Mildly disturbed, Normal, etc.), there is no standardization of their qualitative summary, wherein what one clinician says is “mildly disturbed”, another might call “normal”, depending on the type of cases they are used to seeing.

The present standardization process first changes each reported metric into a true standard metric through the use of a z-score transformation. Then, depending on where the particular score falls among the z-score distribution, it is designated as “improved”, “no change”, or “declined”, based on the previous scores reported.

At step 510, the entered symptoms are categorized into one or more categories 503 including ABI (Acquired Brain Injury), dementia, developmental, and psychological. At step 515, the probabilities of certain specific diagnoses are calculated using a decision tree 129.

Table 1, below, provides an example of the use of differential decision tree 129 to rule out the most common CNS conditions that result in an adult coming to ER for suspected CNS problems. Decision trees 129 are part of the diagnostic template 121 and are used in the process of calculating diagnostic probabilities (step 515, FIG. 5). The present system includes decision trees 129 that cover all CNS disorders of interest, with most of the decision trees covering groups of four to five disorders. Exemplary decision tree 129 in Table 1, below, addresses dementia, depression, and delusions.

TABLE 1
Example Decision Tree

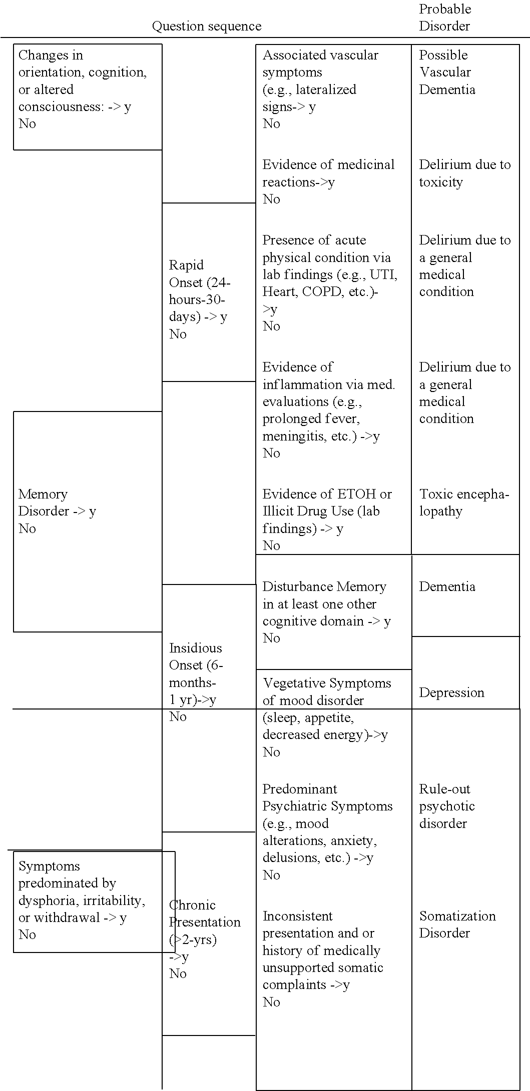

At step 520, a Bayesian probability statistical analysis is performed on the symptom data (using, e.g., processor 101 to execute diagnostic program 160) to determine the degree of probability that a diagnosis indicated through decision tree 129 is accurate, given additional information about the patient and his/her condition (e.g., specific history issues, etc) for each symptom of interest. At step 525, a master diagnostic graph 526 is generated which shows these probabilities for each of the diagnostic results. At step 530, the received and generated diagnostic data is stored in database 111 via cloud 105.

### Cloud-Only Use Example

A patient suffers a traumatic head injury (TBI) that affects his speech and ability to move. Diagnostic program 160 is used to assist in providing treatment. The physician treats the movement disorder via a medication to decrease muscle tone and this is recorded in the program via the private practice PC (personal computer or portable computing device) 110. The patient's parents treat the motor problem by performing range of motion exercises with the patient and this is recorded in the program via a PC 110 in the patient's home. The teacher addresses the problems with various academic maneuvers, which are recorded in the program via a school PC 110, and a speech pathologist sees the patient for language problems and records data into diagnostic program 160 via a PC 110.

Any one of the providers noted above may view what the others are doing and how the patient is progressing from their PC, from information transmitted via computing cloud 105. Treatment may be modified or terminated via program 160, as well.

### Cloud Via Tablet Use Example

Starting with the same scenario as above, add to the problems blood sugar deviations secondary to the traumatic brain injury. The events as described above would follow in this situation as well, with the significant addition of the clinicians need to have daily monitoring of the patient's blood sugar level to gauge the patient's blood sugar treatment. Therefore, this patient may use a tablet 106 or equivalent device to communicate with diagnostic program 160. The patient's parents and other caregivers may still enter data through their personal PCs. In any case, the patient's blood sugar would be monitored several times a day by using a peripheral device that transmits the blood sugar analysis to diagnostic program 160 directly, thus eliminating human error in data entry.

Having described the invention in detail and by reference to specific embodiments thereof, it will be apparent that modifications and variations are possible without departing from the scope of the invention defined in the appended claims. More specifically, it is contemplated that the present system is not limited to the specifically-disclosed aspects thereof.

1. A method for monitoring central nervous system development of a patient comprising:
administering, to the patient, a diagnostic screener comprising a set of age-specific questions to determine one or more central nervous system problems;
repeating the following computer-implemented steps until the monitoring is concluded:
receiving diagnostic input using a CDE template application to accept patient symptoms that match at least one CDE symptom in a list of predetermined symptoms which correspond to NINDS common data elements;
calculating probabilities of certain diagnoses based on the patient symptoms accepted by the CDE template application;
performing statistical analysis of the patient symptoms to determine a set of results including, for each symptom of interest, the probability of a corresponding diagnostic result;
generating a diagnostic graph displaying the set of results; and
generating a standardized, age-appropriate assessment of the central nervous system problems using data from the diagnostic screener.

2. The method of claim 1, wherein the diagnostic input is received from a plurality of clinicians and used to calculate the probabilities of certain diagnoses.

3. The method of claim 1, wherein a standardized language indicating the degree of patient impairment is used to indicate a present status of each of the patient symptoms.

4. The method of claim 1, wherein the diagnostic screener is administered at a school attended by the patient.

5. The method of claim 1, wherein the diagnostic screener provides a snapshot of CNS functioning, indicating CNS-related functions including sensory, motor, perception, memory, cognition, and executive, wherein a range between low average and high average is indicated for the respective functions.

6. The method of claim 1, wherein the diagnostic input is analyzed to generate graphical information including a trend analysis graph indicating efficacy of the treatment over a period of time.

7. The method of claim 6, wherein the trend analysis graph indicates the efficacy of the treatment in terms of categories including “improved”, “stable”, and “declined”.

8. A method for monitoring central nervous system development of a patient comprising:
administering, to the patient, a diagnostic screener comprising a set of age-specific questions to determine a central nervous system problem;
repeating the following computer-implemented steps until the monitoring is concluded:
monitoring, via a peripheral device, one or more biometrics of the patient;
receiving treatment input indicating the type of treatment being received by the patient including treatment for physical, cognitive, behavioral, social, and comorbid conditions;
recording patient symptoms to determine any changes therein since the previously-performed recording step, including changes in areas including physical, cognitive, behavioral, social, and comorbidity;
standardizing any changes in the patient symptoms observed since the previously-performed step of receiving treatment input;
storing the patient symptoms, the set of results, the biometrics, and standardized changes in the patient symptoms in a database; and
generating a treatment graph displaying the changes in the patient symptoms.

9. The method of claim 8, wherein the treatment graph simultaneously displays indications of the patient's blood pressure, blood sugar, spasticity, executive dysfunction, and memory, to provide a holistic picture of the patient's functioning.

10. The method of claim 8, wherein the treatment graph includes input from sources including professionals, non-professionals, and non-medical professionals.

11. The method of claim 8, wherein a standardized language indicating the degree of patient impairment is used to indicate a present status of each of the patient symptoms.

12. The method of claim 8, wherein the standardized language indicates whether the changes in a particular symptom represent improvement, decline, or stability, based on metrics used by a clinician recording the symptom.

13. A method for monitoring central nervous system development of a patient comprising:
repeating the following steps until the monitoring is concluded:
receiving treatment input indicating the type of treatment being received by the patient, wherein the treatment input is constrained, via a computer application, to the categories of physical, cognitive, behavioral, social, and comorbid conditions;
recording patient symptoms to determine any changes observed therein since the previously-performed step of receiving treatment input, including changes in areas including physical, cognitive, behavioral, social, and comorbidity; and
generating a treatment graph displaying the changes in the patient symptoms.

14. The method of claim 13, wherein the treatment graph simultaneously displays indications of the patient's blood pressure, blood sugar, spasticity, executive dysfunction, and memory, to provide a holistic picture of the patient's functioning.

15. The method of claim 13, wherein the treatment graph the graph includes input from sources including professionals, non-professionals, and non-medical professionals

16. The method of claim 13, wherein a standardized language indicating the degree of patient impairment is used to indicate a present status of each of the patient symptoms.

17. A method for monitoring treatment of central nervous system disorders of a patient comprising:
repeating the following steps until the monitoring is concluded:
receiving a plurality of patient symptoms selected from a list of predetermined symptoms which correspond to NINDS common data elements;
measuring the symptoms in standardized units;
recording changes in the symptoms;
standardizing the recorded changes; and
displaying an indication of the standardized changes since the previously-performed step of recording changes.

18. The method of claim 17, wherein a standardized language indicating the degree of patient impairment is used to indicate a present status of each of the patient symptoms.

19. The method of claim 17, wherein the patient symptoms are analyzed to generate graphical information including a trend analysis graph indicating efficacy of the treatment over a period of time; wherein the graph indicates the efficacy of patient treatment in terms of categories including “improved”, “stable”, and “declined”.

20. A method for monitoring central nervous system development of a patient comprising:
administering, to the patient, a diagnostic screener comprising a set of age-specific questions to determine a central nervous system problem;
repeating the following computer-implemented steps until the monitoring is concluded:
receiving diagnostic input using a CDE template to accept patient symptoms that match at least one CDE symptom in a list of predetermined symptoms which correspond to NINDS common data elements;
calculating probabilities of certain diagnoses based on the patient symptoms accepted by the CDE template;
performing statistical analysis of the patient symptoms to determine a set of results including, for each symptom of interest, the probability of a corresponding diagnostic result;
monitoring, via a peripheral device, certain biometrics of the patient;
receiving treatment input indicating the type of treatment being received by the patient including treatment for physical, cognitive, behavioral, and comorbid conditions;
recording any changes in the patient symptoms since the previously-performed recording step, including changes in physical, cognitive, behavioral, and social areas;
standardizing any changes in the patient symptoms;
storing the patient symptoms, the set of results, the biometrics, and the standardized changes in the patient symptoms in a database;
and
generating a diagnostic graph displaying the set of results, and a treatment graph displaying the changes in the patient symptoms.

21. The method of claim 20, wherein the diagnostic input is received from a plurality of clinicians and used to calculate the probabilities of certain diagnoses.

22. The method of claim 20, wherein a standardized language indicating the degree of patient impairment is used to indicate a present status of each of the patient symptoms.
